(19) United States
(12) Patent Application Publication
(10) Pub. No.: US 2026/0115410 A1
(43) Pub. Date: Apr. 30, 2026

(54) Method of Therapeutic Massage Modalities for Enhancing Muscle Relaxation and Mobility

(71) Applicant: Cheryl Smith, Tyler, TX (US)

(72) Inventor: Cheryl Smith, Tyler, TX (US)

(21) Appl. No.: 18/925,330

(22) Filed: Oct. 24, 2024

(51) Int. Cl. A61M 21/02; A61H 33/04; A61H 39/04; A61H 39/06; A61H 99/00; A61M 21/00

(52) U.S. Cl. CPC A61M 21/02; A61H 33/04; A61H 39/04; A61H 39/06; A61H 99/00; A61H 2033/048; A61H 2201/105; A61H 2203/0468; A61H 2205/12; A61M 2021/0016; A61M 2021/0022; A61M 2210/0612

(57) ABSTRACT

A therapeutic massage method that begins with a customized bath followed by client ringing a bell signalling the client's readiness to begin the massage comprising performing a ballet style core massage, performing rock positioning massage, placing a washcloth under a heated rock on the recipient's core to promote relaxation and deep muscle release, applying a magnesium solution to sit on skin while preparing a cup towel and heat pack, placing the cup towel over a treated area, followed by positioning the heat pack contained in the fabric over the treated area, allowing absorption of the magnesium solution into the skin and inhalation treatment using a temperature-adjustable eye pillow pack until the completion of massage session as determined by both therapist and recipient that all areas of concern are addressed to provide enhanced muscle relaxation.

### FIELD OF INVENTION

This invention relates to the field of therapeutic modalities and spa therapy, specifically to techniques and methods designed to enhance muscle relaxation, reduce pain and improve mobility that will usher in changes within the massage industry.

### BACKGROUND OF THE INVENTION

Therapeutic massage and spa treatments are long sought effective methods to alleviate muscle tension, pain, and restricted mobility. Therapeutic modalities refer to the administration of thermal, mechanical, electromagnetic and light energies for therapeutic purposes. They are commonly used by physiotherapists to help their patients/clients achieve therapy goals: pain relief or modulation. Massage and spa modalities encompass a diverse range of therapeutic techniques and treatments designed to promote relaxation, rejuvenation, and overall wellness. These modalities have evolved over centuries, drawing from cultural practices and medical traditions worldwide. Spa modalities also extend beyond massage to include facials, body wraps, scrubs, and specialized treatments like mineral baths, reiki, and sound therapy. Each modality offers unique benefits and is often combined to create customized spa experiences tailored to individual preferences and wellness goals. As the understanding of holistic health continues to evolve, these modalities along with a better parameter of how to time and price these modalities play a crucial role in promoting physical, emotional, and spiritual well-being.

In recent years, the field of massage therapy has continued to grow and evolve with advancements in research, technology, and techniques. It is increasingly integrated into conventional healthcare as a part of holistic treatment plans for a wide range of conditions, from sports injuries to chronic pain management and stress reduction, however, appointment timing slots and separately priced tools or modalities have been rigidly in place for decades, making access to every treatment possible difficult to receive without being charged a much more expensive bill at the end of the service.

In modern times, massage therapy has become recognized as both a complementary and alternative medicinal practice. It is widely practiced in clinical settings, spas, wellness centers, and even hospitals as the therapeutic treatment aimed at enhancing physical, emotional, and mental well-being.

There are various types of known massage and spa modalities including: swedish massage, deep tissue massage, sports massage, hot stone massage, thai massage, aromatherapy massage, reflexology, prenatal massage, infant massage, craniosacral therapy, hydrotherapy, and the like. The benefits of various types of massage modalities are extensive and are supported by various scientific researches. Beyond relaxation, regular massage can help alleviate chronic pain, reduce muscle tension, improve flexibility and range of motion, boost immune function, enhance circulation, promote faster healing of injured tissues, and even support mental health by reducing anxiety and depression.

There are certain known methods of utilizing magnesium and essential oils in massage therapy practices, helping alleviate pain and helping detoxify the body., The integration of heat therapy and the application of magnesium into massage practices have shown promising outcomes in the therapy.

The known massage techniques, while beneficial, often lack the precision and multifaceted approach needed to address complex muscular issues. One recognized challenge in known massage therapy is the difficulty in achieving deep muscle relaxation and precise targeting of specific muscle groups. Additionally, a patient often experiences temporary relief, and requires on-going sessions for sustained benefits or relief. Further, the techniques like deep tissue massage or intense stretching can lead to muscle soreness or even injury, not suitable for certain medical conditions like osteoporosis or recent injuries. Certain techniques, especially those involving deep pressure or stretching, can cause discomfort or pain during the massage session. Aromatherapy and the use of essential oils in massages can trigger allergic reactions in some individuals, especially those with sensitive skin or allergies. Regular massages and spa treatments can be costly, making them inaccessible for some individuals who may benefit from their therapeutic effects.

This necessitates the development of techniques and methods that not only provide immediate relief but also support long-term physical and neurological health of individual to enhance the overall effectiveness of treatments, and also the unchanged way of timing appointments and pricing necessitates a change as well, a change that would better suit the needs of the client, the licensed massage therapist, and spa owners.

### OBJECTIVE OF THE INVENTION

An objective of the present invention is to develop a method of therapeutic modalities for use in massage therapy and spa treatments, aimed at enhancing muscle relaxation, reducing pain, and improving mobility and mental well-being. These therapeutic modalities also address the structural state of clients and those modalities include a pre-massage mineral bath and bell preparation, ballet core and legs massage, rock positioning massage, magnesium and heath pack treatment, foot scrub treatment, eye pillow with inhalation treatment, and big rock core treatment.

Another objective of the present invention is to provide a comprehensive and synergistic approach for precise and effective targeting of muscle groups, allowing for deeper muscle penetration to provide comprehensive muscle relaxation, pain relief by the integration of method of therapeutic modalities.

The objective to a mineral bath massage preparation, which is called bath and bell, is to mentally and physically relax the client in preparation to better receive their massage, thus getting the most therapeutic value possible. Also this bath chamber experience allows the client to participate by choosing their unique customizations such as the color of the light in the bath chamber room, and also choosing what goes into their bath. The interaction builds trust and makes for a more secure feeling to relax in preparation for their massage.

Another objective of the present invention is to ensure that the integrated modalities promote a soothing and comfortable experience, facilitating better communication and individualized care for the recipient, further supporting sustained physical (muscular) and metal (neurological) health through the combined benefits of heat pack, magnesium and aromatherapy, in order to address both immediate symptoms and underlying issues.

Another objective to the present invention is to provide a comprehensive and synergistic approach for precise and effective targeting of muscle groups, allowing for deeper muscle penetration to provide a comprehensive muscle relaxation and pain relief by integration of method of therapeutic modalities and spa treatments. Another objective of the present invention is to allow therapists to choose treatments based on individual modalities needs and preferences, using a variety of tools and techniques to maximize therapeutic outcomes.

Another objective to this invention is to take time limitations and mounting costs of add-ons off both the client and the massage therapist, reducing mental stress, focusing on the job and having freedom to use any tools or add-ons necessary to complete the massage properly, withholding no good thing that would help the client return to structural correctness. This gives way for the therapist to just be able to do the best job possible.

Another objective of this present invention is to ensure a smooth process for the client by good communication skills, introducing each tool and explaining what each tool does before using, making sure it's okay with the client before therapist proceeds. Yet another objective of the present invention is to ensure the safety of implemented methods for a wide range of recipients, with clear guidelines to avoid potential side effects such as bruising or skin irritation, which ensure the efficacy of each modality.

Another objective of the present invention is to ensure the safety of implemented methods for a wide range of recipients, with clear guidelines to avoid potential side effects, such as bruising or skin and irritation, which show the efficiency of each modality and spa treatment, and or customize therapeutic bath.

Another objective to this invention is to allow clients to customize their massage add-ons, by choosing what products they would wish to include in their treatments.

Another objective for this invention is too promote a triple win effect. With all of the above modalities and spa treatments, this gives the client the best situation for receiving everything they need in a massage appointment, this gives the massage therapist the freedom to simply work until a job is complete, and it also helps spa owners develop a reputable business giving the larger percentage of work done to the massage therapist, making money from product sales and bath treatments.

The foregoing and other objects, features, and advantages of the present invention will become readily appearance upon further review of the following summary and detailed description of the preferred embodiment.

### SUMMARY OF THE INVENTION

This section provides a general summary of the disclosure and is not meant to be a comprehensive disclosure of the full scope of all its features.

In an aspect, the present invention provides a method of therapeutic modalities to enhance muscle relaxation, reduce pain, and improve mobility and structural correctness in massage therapy and spa treatments. This aspect of the invention provides either individual or combined effect of several therapeutic modalities such as bath and bell mineral bath experience, ballet core and legs, massage, rock positioning massage, magnesium & heat pack treatments, footscrub treatments, eye pillow with inhalation treatment, and big rock core treatments to achieve superior therapeutic outcomes.

The present invention addresses the above mentioned drawbacks and/or limitations by providing various therapeutic modalities method protocol that not only enhances the immediate benefits of massage therapy but also promotes sustained muscle health and improved overall well-being for the recipients along with structural support.

In an embodiment of therapeutic mineral baths, the method of the bath and bell experience gives clients a sense of security with the very private bath chamber located inside every massage room much like a master bathroom is to a bedroom. It also gives the client an opportunity to be part of the customizing of their bath experience by choosing what bath minerals and farmhouse fresh bath gel goes into their bath. Client also get a sense of relaxation and mental well-being from choosing their light color or colors to enjoy while inside the bath chamber. This unique process creates something that is called that first dip feeling. When the client is done with their bath, they simply get out of the tub, dry off, and proceed to the massage table where they will find a bell on the massage table that they can ring when they are ready for the therapist to come in and being the massage. This whole experience mentally and physically prepares the client to receive optimum massage therapy benefits as the muscles take a better massage with this preparation.

In an embodiment of structural and therapeutic modalities, the method comprising and performing a ballet style core and leg massage works by applying pressure with or without rocks placed under the clients body in specific areas of palpated pain, holding that specific area of palpated pain and then getting client to assemble themselves to specific ballet positions produces relief from muscle tightness and provides a precise and effective way to lessen pain and provide better mobility, causing a better structural layout of the body.

In another embodiment of structural and therapeutic modalities, the method of performing rock positioning massage occurs by placing rocks under recipient's body in specific angles, using gravity and the weight of the recipient to achieve relief by either letting the rock sit there for a short amount of time or slowly moving the clients limbs or body, locating a very exact point of precision, causing that point to twist in between the muscle fibers in such a way that relief is accomplished. This can also be used in the ballet core and legs massages. The rocks are of various shapes and sizes to leverage gravity and weight and are placed under specific muscles to provide targeted relief. The rocks are either placed statically or dynamically based on the recipient's movement to achieve cross fiber deep muscle relaxation. The various shape and size of the rock includes flat, shallow-height rock that minimizes bruising in sensitive areas of the body.

In another embodiment of therapeutic modalities, the method of comprising and preforming a magnesium and heat pack treatment to desired areas adds even further muscular relaxation to the massage session. The magnesium and heat pack therapy are performed by making the magnesium solution with magnesium flakes and water, putting the solution on the client, adding massage cream and or a carrier oil to dilute in case it stings, adding non-cooling essential oils of the client's choice, letting that mixture sit for a minute or two before adding a cup towel over the applied solution area, then finally adding the heat pack to further allow magnesium and or non-cooling essential oils to more deeply penetrate the skin.

In another embodiment of therapeutic treatments, the method of comprising and preforming a foot scrub treatment provides enhanced relaxation and skin care benefits. This unique foot scrub is performed with the recipient lying in the prone position on the massage table with a bolster underneath the ankles; then a towel on top of that bolster stretching the span of the end of the massage table. The therapist wets the recipient's feet with the warm wet washcloth. Then the therapist applies a salt or sugar scrub of the client's choice to the heels and feet, scrubbing the heels with a hot stone; next wetting the feet and wiping the scrub away, then adding moisturizer of the recipient's choice.

In another embodiment of therapeutic treatments, the method of comprising and performing an eye pillow with inhalation treatment helps relax and unbusy the mind while inhaling peppermint, eucalyptus or rosemary and or the like essential oils. The pillow is placed on a washcloth folds over the top of the eye pillow in a “v” shape where the essential oils are added so they don't actually touch the nose but rather hangover right in front of the nose slightly for inhalation purposes. In an embodiment, the essential oils include peppermint, eucalyptus, rosemary, or lavender and the like.

In another embodiment of massage therapy treatments, the method of comprising and performing a big rock core treatment, it allows clients to feel grounded with a heavy stone that is heated and placed on top of the massage sheets on the belly area, also providing a relaxed center, providing either as a preparation for massage or as an after massage treatment to add further relaxing of the abdominal muscles.

In an embodiment, these modalities and treatments are designed to either work together or individually, offering a comprehensive approach to structural therapeutic massage and treatments. These methods of therapeutic massage modalities also facilitate communication between the therapist, feedback, choices, important safety measures, and also ensures that each treatment is tailored to individual needs and preferences while also promoting long-term and neurological support.

Another very important embodiment here is the way the appointment is timed, allowing it to be finished whenever the more corrected structural layout of the body is complete. This is determined by the recipient. When the therapist thinks everything is covered, the therapist will then ask the recipient if there's anything else bothering them that they need to cover. The therapist either continues to work a little more if more problem areas were stated by the recipient or the recipient and the therapist both agree the massages over at that point, and thus time pressures that usually stress the therapist are gone for this complete massage to occur.

Yet in another embodiment of therapeutic treatments, all tools or regular spa add-ons are all inclusive and equated into a flat rate pay system, giving every tool and possible good things available to the therapist to use on the recipient without the pressures of mounting costs. This flat rate pay system is measured in time increments determined by the spa owner. Some spa owners may want to do 15 minutes time increments and charge a preset amount per 15 minutes. Another spa may want to choose 10 minute increments and charge a different amount for every 10 minutes. These are examples, but each spa can determine their time increment and charge per increment, and include any and everything that is necessary per each customized massage treatment. This helps promote a stress-free work environment for not only the therapist and spa owner, but also the recipient. Additionally, massage time does not start until the massage actually begins and does not end until the massage actually ends.

In an embodiment, these modalities are designed to either work together or individually, offering a comprehensive approach to therapeutic massage and spa treatments. The methods of therapeutic massage modalities also facilitates communication between the therapist and the recipient regarding on-going therapies, feedbacks, choices, important safety measures and also ensures that each treatment is tailored to individual needs and preferences, while also promoting long-term muscular and neurological health.

Other features, benefits, and advantages of the present invention will be apparent upon a review of the present disclosure, including the specification, abstract, and claims.

### DETAILED DESCRIPTION OF THE INVENTION

The embodiments herein and the various features and advantageous details thereof are explained more fully with reference to the non-limiting embodiments disclosed in the following description. Numerous variation, changes and substitution may occur to those skilled in the art without departing from the invention. It should be understood that various alternatives to the embodiments of the present disclosure herein may be employed.

At the outset, for ease of reference, certain terms used in this application and their meanings as used in this context are set forth. To the extent a term used herein is not defined below, it should be given the broadest definition persons in the pertinent art have given that term as reflected in at least one printed publication or issued patent. Further, the present methods and techniques are not limited by the usage of the terms used in application, as all equivalents, synonyms, new developments, and terms or techniques that serve the same or a similar purpose are considered to be within the scope of the present claims.

The articles “a” and “an” as used herein mean one or more when applied to any feature in embodiments of the present invention described in the specification and claims. The use of “a” and “an” does not limit the meaning to a single feature unless such a limit is specifically stated. The article “the” preceding singular or plural nouns or noun phrases denotes a particular specified feature or particular specified features and may have a singular or plural connotation depending upon the context in which it is used. The adjective “any” means one, some, or all indiscriminately of whatever quantity.

The present invention encompasses a series of therapeutic modalities aimed for enhancing the effectiveness of massage therapy and spa treatments. The series of therapeutic modalities include bath and bell mineral bath treatment, ballet core and legs massage, rock positioning massage, magnesium and heatpack treatments, foot scrub treatment, eye pillow with inhalation treatment, and big rock core treatment. These modalities and treatments either work individually or synergistically to provide targeted muscular relief, improve mobility, support structural correctness, and promote overall relaxation and well-being.

In an embodiment, the method includes performing a ballet style core and leg massage where a pressure and movement technique is applied to stretch the muscles of the body part based on ballet positions to get relief from muscle tightness and provide precise & effective way for less pain and better mobility of the stressed body part. The therapeutic mineral treatment call bath and bell include several details to promote not only physical relaxation but mental, emotional security relaxation as well. The process begins by the therapist going into the bath chamber and showing the recipient the process of choosing their own bath salts, and bath gel. The therapist allows the recipient to fill up a bowl with the salts they desire, and then the therapist puts the bowl of salts in the bath that would be currently being drawn. Then the therapist allows the client to choose with bath gel they would like to put into their bathwater. The recipient hands the therapist product and the therapist puts the product in the tub. Then the therapist allows the client to pick what color LED lights they would wish to have on in the bath chamber. The bath chamber will always be located in each massage room just like a master bathroom has a master bedroom, except the bath chamber is strictly for therapy baths. Nestled in privacy, the bath chamber gives the client a sense of security. After the desired ingredients are in the bath, the therapist instructs the recipient to add a little bit of cold water if they need it, but not to fill past a certain point. The therapist shows the client where their towel is and where they can put their clothing. Then the therapist instructs the recipient on how to get in and out of the tub and makes a few stretch suggestions for the client to do while in their bath chamber experience. All this is done while the recipient is still clothed. All instructions are given before the therapist leaves the room and is never in the bath chamber while the recipient is getting undressed and getting into the tub and having their bath experience. When the perfect bath is drawn and the recipient gets into the bath, there is something personalized and wonderful upon the first moments in the bath. After all the instructions are given from the therapist to the recipient, then the therapist says when you are ready to get out of your bath, simply get out, dry off, and proceed to the massage table where you will find a bell attached to the massage table. The therapist tells the client to ring the bell when they are ready for the therapist to enter and begin the massage. The entire bath and bell experience is a great preparation physically and mentally for the client to not only have a smooth process but a wonderful head start on muscle relaxation and mental relaxation prior to their massage. All ballet positions or movements performed are done while the client is lying on the massage table. The movement technique of the ballet style core and leg massage can be selected from the group comprising: plies, tendus, degages, rond de jambe, battement tendu, battement fondu, battement frappe, grand battement, adagio, and port de bras, wherein the plies technique comprises bending of leg with the knees turned outwards; the tendus technique comprises extending the leg along the table in various directions while maintaining turnout and pointed toes, the degages technique is similar to tendus but involves lifting the foot a few inches off the table while maintaining contact with the ground, the rond de jambe technique comprises moving the legs from the hip circularly while maintaining turnout, the Battement Tendu technique comprises a combination of tendus and battements and extending the leg sharply from the hip to a pointed position on the table or slightly above it, the battement fondu technique comprises bending and straightening the supporting leg while performing the tendu or degage with the working leg, the Battement Frappe technique comprises striking the table with a ball of the foot, in a flexed position, the Grand Battement technique comprises lifting the leg high in air, while keeping it straight and maintain turnout, the port de bras technique comprises gracefully moving the arms in coordination with the rest of the body. The applied pressure depends upon the type of movement technique selected. The applied pressure and movement of various body parts enhances precision and effectiveness of muscle targeting in order to achieve muscle relaxation.

In an embodiment, the method includes performing a ballet style core and leg massage where pressure, movement and even gravitational techniques are used to stretch the muscles of the targeted body part with slow assisted movements involving specific ballet positions while the recipient is on the massage table. This method delivers relief from muscular tightness and provides a precise and effective way for decreasing pain and producing better mobility. Structural alignment often naturally occurs during some of these modalities. In one embodiment and particular modality, the recipient lies flat in the supine position (face up) on the massage table while the therapist is seated along one side of the recipient's abdominal area. The therapist takes the massage cream and a warm stone and reaches underneath the recipients back and sciatic areas, massaging from back around front of the abdomen. The therapist looks for the tightest areas and takes a mental note of where the structural alignment of the client seems to be off from the correct state of structural alignment. For instance, the recipient may have a group of muscles that are very tight, causing a structural problem in the recipient's daily life; problems such as sitting or walking. Once the problem area is identified by the therapist, then a modality determination can be made. For instance, the recipient may require a specific rock to be placed under the back or under the hip. The therapist states to the recipient that they will hold the rock underneath the back or the hip and have the recipient slowly perform a ballet coupe position. The therapist can feel the muscles under the rock being stretched. Then the therapist may tell the client to come all the way up to a posse position. Quite often with the rock under the right place, and the recipient slowing moving into these ballet positions, their structural alignment seems to fall into place. Part of this type of movement has to do with the gravity of the client's body weight coming down on the rock while the movements are being performed. Ballet positions such as first position or second position, couple, posse, arabesque, and the like are used. Each recipient will have a different set of problems and the therapist has to determine what shape of rock to put under which area of the body while in the ballet positions are performed. Not every ballet position performed requires a rock underneath the client. Sometimes simply massaging while the ballet movements are performed does the job. These movements can be performed in the supine position, the side laying positions and even the prone position, all on the massage table. This specialized massage targets the core and leg muscles, focusing on enhancing flexibility, strength, and alignment that are the key for healthy posture of the core. It combines deep tissue techniques with stretches to relieve tension and improve muscle performance. The combination of deep tissue massage, myofascial release, and trigger point therapy work on muscle groups effectively that aids in significant relief from muscle pain and provide range of motions.

In another embodiment, the method comprises performing a rock positioning massage where rocks of various shapes and sizes are placed strategically under the recipient's body at a predefined angle based on gravity, recipient's weight, and specific muscles needing relief. In particularly the rocks are placed between specific muscle fibers needing relief. It takes feedback regarding placement of the rocks on the exact point of muscle needing relief. If not placed appropriately, adjust the positioning of rocks at correct site based on the feedback from the recipient. The predefined angle depends upon the posture of the body and can be of any angle known to a person skilled in the art. The strategic placement of the rocks of various shapes and sizes leverage gravity and recipient's weight under specific muscles so that the rock may get in between muscle fibers to balance energy flow and provide possible relief from pain, causing muscles to relax by releasing tension and giving better mobility and function to the recipient's body. In an embodiment either leave the rock in place until the recipient is comfortable for it to be removed, or gradually have the recipient change positions so that the rock can work its way between muscle fibers (cross-fibers). In an embodiment, the rock positioning may be stationary or dynamic depends upon the change or movement of body posture or position of the recipient, as the change in position allows the rock to get in and twist in between muscle fibers to facilitate precise targeting of muscle tension in order to provide deep muscle penetration and relaxation. In an embodiment, flat, shallow-height rocks are used that minimizes bruising in sensitive areas of the body, especially when placed under areas of upper back or usually in the hip and glute areas.

In yet another embodiment, the method includes performing a magnesium and heat pack treatment where magnesium flakes are dissolved in water, optionally with or without essential oils (no peppermint or other cooling oils) and apply a solution of magnesium on the desired areas of the body such as back and neck. If the magnesium concentrated form stings on the body, dilute it a bit either with massage oil or massage cream, allowing the magnesium solution to sit on skin briefly while preparing a cup towel and heat pack; placing the cup towel or a thin fabric larger than the heat pack over a treated area, followed by positioning the heat pack contained in the fabric over the treated area; allowing an absorption of the magnesium solution into the skin until the completion of massage session to provide even further enhanced muscle relaxation.

This magnesium, oil, and heat pack treatment can be added on various parts of the body while the therapist works other areas, creating optimum usage of therapy and time. Magnesium supports the muscular and neurological systems of the body, helping to alleviate pain and bring relaxation to nerves.

In addition, the essence of the magnesium in association with heat pack therapy lies in the fact that in the present method, the application of heat aids in driving the magnesium into the skin, promoting muscle relaxation on the target areas of the body and also support muscular and neurological functions in enhancing the overall therapeutic effect. Further, the heat pack complements the therapy by promoting circulation and easing muscle stiffness, offering a holistic approach to muscle relaxation and pain relief.

In an embodiment, the method includes performing spa foot scrub treatment with positioning the recipient's face down on a massage table. Place a bolster pillow under recipient's ankles and cover it with a towel that extends close to the end of the table, ensuring it's larger than the bolster itself. A warm washcloth is moistened and gently applied to the recipient's feet, preparing them for the exfoliation process. The recipient chooses what salt or sugar scrub they prefer when the therapist allows them to choose by smelling. A body or foot scrubber, a warm stone, is used to briskly (for callused skin) or gently exfoliate the heels and soles of the recipient's feet, effectively removing or exfoliating dead skin cells. The scrub is then wiped away from the feet using the warm washcloth. It may be necessary to rinse the washcloth periodically and ensure all scrub granules are thoroughly removed. Once the feet are clean and exfoliated, a balm or moisturizer of recipient's choice is applied to nourish and hydrate the skin. This step allows the moisturizing agent to absorb fully into the skin, providing enhanced relaxation and skin care benefits throughout the remainder of the massage session. The method further removes the dead skin cells, soften rough areas, and stimulate circulation, leaving the feet feeling rejuvenated and smooth in order to provide detailed foot care so that over all relaxation can be enhanced.

In another embodiment, the method includes performing an eye pillow with inhalation treatment using a temperature-adjustable eye pillow pack placed on a washcloth. The temperature-adjustable eye pillow pack can be warmed, chilled, or kept at room temperature. An additional part of the washcloth is folded over the top of the eye pillow pack. The pack is then positioned over the recipient's eyes, with the folded washcloth covering its top and folded sides shaped into a V-shape to rest gently over the recipient's nose without touching it. The recipient selects an essential oil such as peppermint, eucalyptus, rosemary, or lavender and the like. This chosen oil is applied to the V-shaped washcloth for inhalation during massage to enhance the sensory experience and or help clear sinuses. The eye pillow pack remains in place over the eyes throughout the massage session and can be removed either immediately after or according to the recipient's preference. The essence of the eye pillow lies in the fact that it block out light, reduce eye strain, and promote eye relaxation and the inhalation of the aroma from essential oil or soothing scents infused with eye pillow aids in calming the mind, enhancing the sensory experience and overall therapeutic relaxation.

In another embodiment, the method includes performing a big rock core treatment. In this treatment, a very large and heavy river stone about the size of one's stomach cavity can be used to provide a grounding effect, and also a continued relaxing of the core muscles with the weight and heat of the rock. In this treatment, the therapist can put magnesium and essential oils on the belly prior to adding the rock on top of the massage sheets and massage blanket. The therapist can also add a cloth on top of the rock, so that recipient doesn't burn their hands on the heated rock. The rock can remain in place while the therapist works on other parts of the body. This is another way to provide multiple things at one time so the recipient is provided optimum relaxational results, again utilizing the most of the time of treatment. The stone must be removed at the end of this supine positioned massage treatment.

In an embodiment, the washcloth is placed on top of the heated stone to facilitate recipient's comfort, allowing them the option to rest their hands on it if desired. This barrier prevents direct contact between the recipient's hands and the hot stone surface to promote relaxation and deep muscle release to get relief from pain, causing muscles to relax and giving better mobility and function to the recipients.

In an embodiment, the effectiveness of the therapy depends upon adjusting therapy plans based on received feedback, outcomes and preferences of the recipient, soliciting subjective feedback on pain relief, relaxation achieved, and overall satisfaction with the therapeutic outcomes to adjust the therapeutic modality plan. The adjustment of therapy plans modifies the selection of massage techniques, intensity levels, and duration of therapy sessions based on user-reported outcomes and preferences.

In another embodiment, a business model for massage and spa services comprises an all-inclusive pricing structure for any type of massage or spa treatment is included in a single flat rate and the flat rate is applied to the chosen time increment set by the business owner.

In another embodiment, the business owners can select their preferred time preferred time increments, such as every 10, 15, or 20 minutes. The examples rates include $25.00 per 15 minutes or $35.00 per 20 minutes and the price per increment is solely determined by the business owner, not exceeding highest price approved by business owner.

In another embodiment, the business model offers real time charging in which the billing period starts when the massage begins and ends when the massage concludes.

In another embodiment, the business model includes over whenever complete option in which the massage session ends when both the therapist and the client agree that all areas of concern have been addressed, a predetermined rate per additional minute is applied if the session concludes at an odd time not aligned with the set increments, if the session is set at $25.00 per 15 minutes and ends at 1 hour and 5 minutes, the business owner may charge $2.00 per extra minute or whatever price the owner determines is most suitable within reason, not exceeding the highest price per minute set by business owner.

In another embodiment, the business owners can pre-set blocks of time for the “over whenever complete” sessions. The optional fees may be applied for reserving these blocks of time or a fixed rate can be set for the entire session, potentially providing cost savings for clients.

In yet another embodiment, the business model includes communication protocol in which clients are informed about the all-inclusive service and any specific choices made by the business owner. Moreover, clients agree to allow the therapist to use their professional judgment for the best results, and are allowed to request any add-on that the spa offers.

In another embodiment, clients who were previously told that the massages are all finished whenever they are complete are now treated for a chunk of time that is blocked off for their appointment. Typically, three hours are blocked off for each appointment. However, the clients can request a more limited time amount if the spa owner chooses to allow that option. Although the typical time amount to be blocked off for an appointment is three hours, the spa owner may wish to choose a slightly different amount of time to block off allowing the business owner to fit more morning appointments, making time for lunch and rest and two evening appointments. This embodiment makes room for at least four appointments possibly five per day. This approach lets the client simply book a massage without having to worry about how long they are going to be in treatment. Additionally, the client previously knows that they get any and all add-ons and tools necessary with no extra monetary charges. This approach takes pressure off the client and the therapist, giving complete freedom to do whatever needs to be done to the utmost for the most comprehensive professional care the industry has to offer.

In another embodiment, the therapists advise clients to communicate any discomfort or preferences regarding temperature, pressure, or positioning and clients are encouraged to take deep breaths to aid muscle relaxation.

In an embodiment, therapists inform clients before using any tools (e.g., cupping cups, hot stones) and provide a brief explanation of their purpose and simultaneously consent is obtained before applying any substances such as essential oils or magnesium.

Overall, the therapists throughout the session maintain open communication to ensure client comfort and satisfaction. The therapist also explains any new techniques or tools being introduced during the massage.

The invention provides a comprehensive care through the use of a series of therapeutic modalities, including but not limited to, the application of heat, magnesium, aromatherapy, and specialized positioning techniques. Each of these treatments either individually or in combination offer unique benefits aimed at promoting physical relaxation, mental calmness, and overall well-being, catering to specific needs and preferences of recipient's seeking holistic health, wellness experiences and structural support.

Although the present invention has been illustrated and described herein with reference to preferred embodiments, it will be readily apparent to those of ordinary skill in the art that other embodiments may perform similar functions and/or achieve similar results. All such equivalent embodiments are within the spirit and scope of the present invention, are contemplated thereby, and are intended to be covered by the following claims.

The advantages set forth above, and those made apparent from the foregoing description, are efficiently attained. Since certain changes may be made in the above construction without departing from the scope of the invention, it is intended that all matters contained in the foregoing description as illustrative and not in a limiting sense.

It is also to be understood that the following claims are intended to cover all the generic and specific features of the invention herein described, and all statements of the scope of the invention that, as a matter of language, might be fall there within.

I/We claim:

1. A method of therapeutic massage modalities, the method comprising:
a customized therapeutic bath experience based on individualized care for the recipient in a bath chamber room;
a bell on the massage table that a recipient can ring when they are ready for the therapist to come in and begin performing a ballet style core massage by applying pressure and movement techniques based on ballet positions determined by area of muscles that need relief;
performing rock positioning massage by placing rocks under recipient's body at a predefined angle based on, recipient's weight, specific muscles needing relief, wherein the placing includes positioning the rocks between specific muscle fibers;
applying a rock core by placing a washcloth under a heated rock on the recipient's core to promote relaxation and deep muscle release;
applying a magnesium by dissolving the magnesium flakes in water, optionally with warming essential oils, to desired areas;
allowing the magnesium solution to sit on skin briefly while preparing a cup towel and heat pack;
placing the cup towel or a thin fabric larger than the heat pack over a treated area, followed by positioning the heat pack contained in the fabric over the treated area;
allowing an absorption of the magnesium solution into the skin until the completion of massage session as determined by both therapist and recipient that all areas of concern are addressed to provide enhanced muscle relaxation.

2. The method of claim 1, wherein performing the rock positioning massage between the muscle fibers promotes potential relief from pain, and provides muscle relaxation, and improved muscle mobility.

3. The method of claim 1, wherein the rocks are of various shapes and sizes and are placed under specific muscles to provide targeted relief.

4. The method of claim 1, wherein the rocks are placed statically or dynamically based on the recipient's movement to achieve cross fiber muscle relaxation.

5. The method of claim 3, wherein various shape and size of the rock includes flat, shallow-height rock that minimizes bruising in sensitive areas of the body.

6. The method of claim 1, wherein applying pressure and movement techniques in the ballet style core massage enhances precision and effectiveness of muscle targeting.

7. The method of claim 1 further comprises a foot scrub treatment, comprising:
positioning the recipient's face down on a massage table;
placing a bolster pillow under the recipient's ankles and covering it with a towel that extends close to the end of the massage table;
wetting a warm washcloth and applying it to the recipient's feet;
applying a body or foot scrubber or a hot stone to briskly scrub salt or sugar solutions onto the heels and soles of the recipient's feet to gently but firmly exfoliate the dead skins;
wiping the scrub away from the feet using the warm washcloth; and
applying a balm or moisturizing agent of choice to the feet after ensuring all scrub granules are removed;
allowing full absorption of the moisturizing agent into the skin until the completion of the massage session as determined by both therapist and recipient that all areas of concern are addressed to provide enhanced relaxation and skin care benefits.

8. The method of claim 1 further comprises
placing a temperature adjustable eye pillow pack over a washcloth;
placing the washcloth under the selected eye pillow pack, with the extra part of the washcloth folded over the top of the eye pillow pack;
placing the eye pillow pack on the recipient's eyes using the folded washcloth portion to cover the top of the eye pillow pack, folding each side into a V-shape that sits over the recipient's nose without touching it;
consulting with the recipient to select an essential oil for inhalation;
applying the consulted essential oils to the V-shaped washcloth;
allowing the eye pillow pack to remain placed on the eyes positions until the completion of the massage session; and
removing the eye pillow pack after completing the massage session as determined by both therapist and recipient that all areas of concern are addressed to provide enhanced relaxation and sensory experience.

9. The method of claim 1 and 7, wherein the essential oils are selected from peppermint, eucalyptus, rosemary or lavender oil.

10. The method of claim 1, further comprising
placing a heavy, heated, and flat stone on the washcloth;
placing the washcloth containing the heavy, heated, and flat stone on the recipient's core strategically, wherein the washcloth protects belly from direct stone heat and provide gentle pressure and heat;
optionally, applying magnesium to the belly part of the core before placing the heavy, heated, and flat stone on the core;
allowing stone to remain stationary in its placed position until the completion of the massage session; and
removing the stone after completing the massage session as determined by both therapist and recipient that all areas of concern are addressed to provide grounding and additional heat therapy to the recipient for achieving muscle stability and relaxation.
